(19) United States
(12) Patent Application Publication
(10) Pub. No.: US 2004/0054257 A1
(43) Pub. Date: Mar. 18, 2004

(54) Deflection mechanism for a surgical instrument, and method of use

(76) Inventor: Joe D. Brown, Panama City, FL (US)

Correspondence Address: BACON & THOMAS, PLLC, 625 SLATERS LANE, FOURTH FLOOR, ALEXANDRIA, VA, 22314

(21) Appl. No.: 10/449,533

(22) Filed: Jun. 2, 2003

Related U.S. Application Data
(60) Provisional application No. 60/384,161, filed on May 31, 2002.

(51) Int. Cl. A61B 1/01

(52) U.S. Cl. 600/151; 600/146

(57) ABSTRACT

A mechanism and method of deflecting a surgical instrument involves use of a shape-memory alloy to control deflection, but is not necessarily dependent on the temperature of fluid in the working channel of an endoscope, catheter, or similar instrument through which the surgical instrument is inserted into a patient. The deflection mechanism may be made of a material such as spring steel that remains in a deflected position at room temperatures, and that is affixed to the instrument so as to normally cause bending of the instrument. In that case, the surgical instrument may be forced into an undeflected position, inserted into a sheath that holds the instrument in the undeflected position, and permitted to resume the deflected position by extending the surgical instrument and corresponding deflection mechanism out of the end of the sheath. Straightening of the instrument can then be carried out simply by retracting the instrument and deflection mechanism back into the sheath. Alternatively, the sheath may be used with a deflection mechanism having a critical temperature higher than room temperature but less than the temperature of the human body, in which case the sheath again prevents bending until the surgical instrument and deflection mechanism are extended out of the sheath.

### BACKGROUND OF THE INVENTION

1. Field of the Invention

This invention relates to a mechanism and method for deflecting a surgical instrument, and in particular to improvements or modifications of the mechanism and method disclosed in copending U.S. patent application Ser. No. 10/153,895, filed May 24, 2002 by Joe D. Brown and also entitled “Deflection Mechanism For A Surgical Instrument, Such As A Laser Delivery Device And/Or Endoscope.” The copending application is incorporated herein by reference.

2. Description of Related Art

The above-cited copending application discloses a deflection mechanism for the distal end of a surgical instrument inserted into the working channel of an endoscope, for the shaft of the endoscope itself, and/or for a similar instrument or device such as a catheter, in which bending of the instrument or shaft is carried out by means of a shape memory alloy structure, such as a NITINOL tube, having the properties of being almost as flexible as the fiber alone at low temperatures, and of reverting to a curved shape at higher temperatures, with a radius of curvature less than or equal to the minimum bend radius of the endoscope. According to a preferred embodiment of the invention described in the copending application, the transformation temperature of the shape memory alloy is selected to be slightly higher than body temperature so as to bend and thereby deflect the end of the shaft when a fluid having a temperature greater than or equal to the transformation temperature is supplied to the working channel.

Although the use of the working fluid to control deflection of the shape memory alloy is believed to be the most practical way of implementing the invention, it is also possible to control deflection by means other than the working fluid. The present application offers an alternative way of achieving deflection, permitting use of a memory tube whose is achieved independently of the temperature of fluids in the working channel of the endoscope, rather than dependent of the fluid temperature as in the preferred embodiments described in the copending application.

### SUMMARY OF THE INVENTION

It is a accordingly an objective of the invention to provide a mechanism and method of deflecting a surgical instrument of the type described in the above-cited copending application, but which is not necessarily dependent on the temperature of fluid in the working channel of an endoscope, catheter, or similar instrument.

This objective is accomplished, in its most general form, by providing a surgical instrument deflection mechanism having shape memory characteristics that cause bending of the instrument in a predetermined manner, but in which the bending of the deflection mechanism may occur either before or during surgery, for example by inserting the surgical instrument into an endoscope, catheter, or similar instrument.

For example, the deflection mechanism may be made of a material such as spring steel that remains in a deflected position at room temperatures, and that is affixed to the instrument so as to normally cause bending of the instrument. In that case, the surgical instrument may be forced into an undeflected position, inserted into a sheath that holds the instrument in the undeflected position, and permitted to resume the deflected position by extending the surgical instrument and corresponding deflection mechanism out of the end of the sheath. Straightening of the instrument can then be carried out simply by retracting the instrument and deflection mechanism back into the sheath.

Alternatively, the sheath may be used with a deflection mechanism having a critical temperature higher than room temperature but less than the temperature of the human body, in which case the sheath again prevents bending until the surgical instrument and deflection mechanism are extended out of the sheath.

Because the sheath will obstruct fluid flow in the working channel of an endoscope, it may also be desirable to enable retraction of the sheath following extension and deflection of the surgical instrument and deflection mechanism out of the sheath.

It should be noted that the mechanism and method described in the copending application are also not limited to working fluid dependent deflection. The specific working fluid dependent deflection described in the copending application is simply a preferred embodiment. As a result, it will be appreciated that the principles of the present invention may be applied to any of the embodiments described in the copending application, including any of the variations of those embodiments, and that the principles described herein may be further varied without departing from the scope of the invention.

### BRIEF DESCRIPTION OF THE DRAWINGS

FIGS. 1-6 are schematic diagrams showing side views of the deflection mechanism of the present invention.

### DETAILED DESCRIPTION OF THE PREFERRED EMBODIMENTS

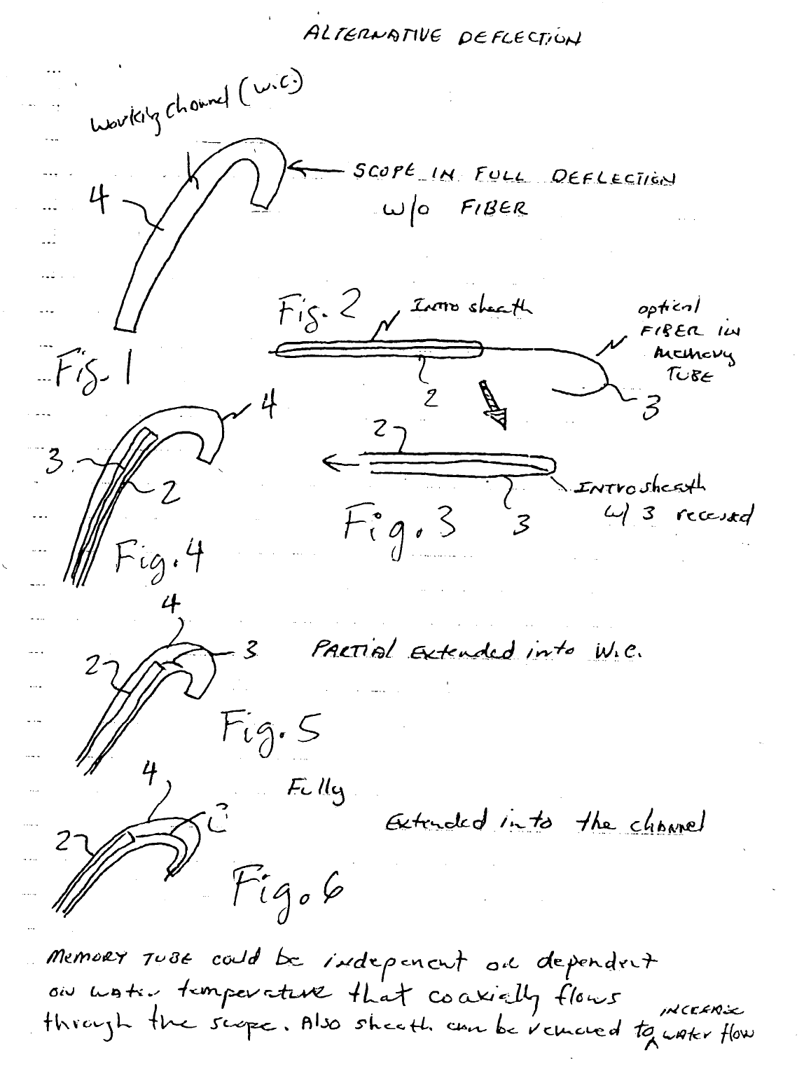

As illustrated in FIGS. 1-6, the deflection mechanism includes a surgical instrument, which may be a laser delivery fiber of the type described in the copending application, positioned in a shape memory tube 3.

As shown in FIG. 2, the shape memory tube 3 is pre-bent over a predetermined angle, such as −180°, to assume a deflected position at normal temperatures, including room temperature and body temperature. A possible material for the tube 3 is spring steel, though the invention is not limited to a particular material.

The shape memory tube 3 is then inserted into a sheath 2. When the shape memory tube 3 is fully retracted into the sheath, as illustrated in FIG. 3, the tube 3 is forced into an undeflected position, and when the shape memory tube extends out of the sheath 2, as illustrated in FIG. 2, it returns to the pre-bent, deflected, position.

In order to use the sheath and deflection mechanism illustrated in FIGS. 2 and 3, the retracted tube 3 and sheath 2 are inserted into the working channel 4 of an endoscope, catheter, or similar instrument, as illustrated in FIGS. 1 and 4. At this time, the endoscope or similar instrument may already have been deflected using a wire or shape memory deflection mechanism of the type described in the copending application.

Once the tube 3 reaches the distal end of the endoscope working channel 4, the surgeon pushes the tube out of the sheath 2, as illustrated in FIGS. 5 and 6, and the tube 3 resumes its pre-bent angle, thereby deflecting the surgical instrument or fiber positioned within the tube. To facilitate fluid flow in the working channel 4, the sheath 2 may then be withdrawn from at least the distal end of the working channel.

Having thus described a preferred embodiment of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with claims to be added upon conversion of this provisional application into a regular U.S. patent application.

What is claimed is:

1. A deflection mechanism for a surgical instrument, comprising a shape memory alloy arranged to be deflected away from a predetermined shape upon placement in a working channel of a device through which the instrument is inserted into a patient, and to resume said predetermined shape when said shape memory alloy is moved out of said working channel.

2. A deflection mechanism as claimed in claim 1, wherein said shape memory alloy possesses said predetermined shape at room temperature and is arranged to normally cause bending of the instrument, and wherein said instrument is forced into an undeflected position upon placement into the device, and caused to resume a bent position upon extension of an end of the instrument out of the device.

3. A deflection mechanism as claimed in claim 2, wherein said instrument is again forced into said undeflected position upon retraction of the instrument back into the device during surgery.

4. A deflection mechanism as claimed in claim 2, wherein said shape memory alloy is made of spring steel.

5. A deflection mechanism as claimed in claim 2, wherein said device is an endoscope.

6. A deflection mechanism as claimed in claim 2, wherein said device is a catheter.

7. A deflection mechanism as claimed in claim 1, wherein said shape memory alloy has a critical temperature higher than room temperature but less than a body temperature of a patient.

8. A method of deflecting a surgical instrument including a shape memory alloy that causes the surgical instrument to have a predetermined shape, comprising the steps of:
a. deflecting the surgical instrument away from the predetermined shape by placing the surgical instrument in a working channel of a device through which the instrument is inserted into a patient; and
b. causing the surgical instrument to resume said predetermined shape when said shape memory alloy is moved out of said working channel.

9. A method as claimed in claim 8, wherein said shape memory alloy possesses said predetermined shape at room temperature and is arranged to normally cause bending of the instrument, and wherein said instrument is forced into an undeflected position upon placement into the device, and caused to resume a bent position upon extension of an end of the instrument out of the device.

10. A method as claimed in claim 9, wherein said instrument is again forced into said undeflected position upon retraction of the instrument back into the device during surgery.

11. A method as claimed in claim 8, wherein said shape memory alloy has a critical temperature higher than room temperature but less than a body temperature of a patient.
